SEQUENCE LISTING (40 sequences)

SEQ ID NO: 1 - DNA, 20 bp, Artificial Sequence
  Synthetic primer
tgcacacttggacagcattt

SEQ ID NO: 2 - DNA, 22 bp, Artificial Sequence
  Synthetic primer
gactccatctccttcctttctt

SEQ ID NO: 3 - DNA, 21 bp, Artificial Sequence
  Synthetic primer
aaactgatgctcttcaaaggc

SEQ ID NO: 4 - DNA, 20 bp, Artificial Sequence
  Synthetic primer
agtggttgtcttagcttgca

SEQ ID NO: 5 - DNA, 23 bp, Artificial Sequence
  Synthetic primer
ccagagagaaagaatcaacagga

SEQ ID NO: 6 - DNA, 22 bp, Artificial Sequence
  Synthetic primer
agatctcttcctccaaactgca

SEQ ID NO: 7 - DNA, 20 bp, Artificial Sequence
  Synthetic primer
agggacatggatgaagctgg

SEQ ID NO: 8 - DNA, 23 bp, Artificial Sequence
  Synthetic primer
tgagtaagtcatagaggaggtcg

SEQ ID NO: 9 - DNA, 20 bp, Artificial Sequence
  Synthetic primer
aagctcagagaggggaactg

SEQ ID NO: 10 - DNA, 20 bp, Artificial Sequence
  Synthetic primer
agtgcttcccctgctcaata

SEQ ID NO: 11 - DNA, 20 bp, Artificial Sequence
  Synthetic primer
cattttgagagtgccccgag

SEQ ID NO: 12 - DNA, 20 bp, Artificial Sequence
  Synthetic primer
aagaggccctggtgacttag

SEQ ID NO: 13 - DNA, 21 bp, Artificial Sequence
  Synthetic primer
ggtgaacaaaaggaccttgga

SEQ ID NO: 14 - DNA, 20 bp, Artificial Sequence
  Synthetic primer
agcaatttgtgaggccaagg

SEQ ID NO: 15 - DNA, 20 bp, Artificial Sequence
  Synthetic primer
gagtgaacggatggtggatg

SEQ ID NO: 16 - DNA, 20 bp, Artificial Sequence
  Synthetic primer
ggggacacacacagaaaagg

SEQ ID NO: 17 - DNA, 20 bp, Artificial Sequence
  Synthetic primer
gcaaatgggcaattgagtgt

SEQ ID NO: 18 - DNA, 20 bp, Artificial Sequence
  Synthetic primer
tccagcctacatccacatct

SEQ ID NO: 19 - DNA, 20 bp, Artificial Sequence
  Synthetic primer
gaccctgtcctagccttctt

SEQ ID NO: 20 - DNA, 18 bp, Artificial Sequence
  Synthetic primer
agcctcagtgactgccag

SEQ ID NO: 21 - DNA, 18 bp, Artificial Sequence
  Synthetic primer
cagagttccggggttggg

SEQ ID NO: 22 - DNA, 19 bp, Artificial Sequence
  Synthetic primer
ggccagcctgttttcttgc

SEQ ID NO: 23 - DNA, 20 bp, Artificial Sequence
  Synthetic primer
cagatcacgaggtcaggagt

SEQ ID NO: 24 - DNA, 20 bp, Artificial Sequence
  Synthetic primer
gtggccagaacttccaacac

SEQ ID NO: 25 - DNA, 20 bp, Artificial Sequence
  Synthetic primer
accagatctccaacaggaca

SEQ ID NO: 26 - DNA, 18 bp, Artificial Sequence
  Synthetic primer
gagcttcctcggcaccag

SEQ ID NO: 27 - DNA, 20 bp, Artificial Sequence
  Synthetic primer
ctagcttgccattctgtgcc

SEQ ID NO: 28 - DNA, 22 bp, Artificial Sequence
  Synthetic primer
accttagaacacacccaattca

SEQ ID NO: 29 - DNA, 20 bp, Artificial Sequence
  Synthetic primer
ggctgtgtctctaagtggca

SEQ ID NO: 30 - DNA, 20 bp, Artificial Sequence
  Synthetic primer
agtttcactcttgttgccca

SEQ ID NO: 31 - DNA, 20 bp, Artificial Sequence
  Synthetic primer
gcagcaccatctttcacagt

SEQ ID NO: 32 - DNA, 20 bp, Artificial Sequence
  Synthetic primer
aaggaaggagagaggtagca

SEQ ID NO: 33 - DNA, 20 bp, Artificial Sequence
  Synthetic primer
atgactttgcgcttcaggac

SEQ ID NO: 34 - DNA, 20 bp, Artificial Sequence
  Synthetic primer
agctttgccaatgttgtcca

SEQ ID NO: 35 - DNA, 21 bp, Artificial Sequence
  Synthetic primer
ggtacctggaaatgacactgc

SEQ ID NO: 36 - DNA, 19 bp, Artificial Sequence
  Synthetic primer
tgattgagtcaccggcatg

SEQ ID NO: 37 - DNA, 20 bp, Artificial Sequence
  Synthetic primer
tcgtaatttccaggccctgt

SEQ ID NO: 38 - DNA, 20 bp, Artificial Sequence
  Synthetic primer
agatcaccccattgctctcc

SEQ ID NO: 39 - DNA, 22 bp, Artificial Sequence
  Synthetic primer
ggagacagagattacatgggtt

SEQ ID NO: 40 - DNA, 20 bp, Artificial Sequence
  Synthetic primer
tggttcaaatcctgcgtctg

(19) United States
(12) Patent Application Publication
(10) Pub. No.: US 2024/0117445 A1
(43) Pub. Date: Apr. 11, 2024

(54) Macrohaplotypes for Forensic DNA Mixture Deconvolution

(71) Applicant: UNIVERSITY OF NORTH TEXAS HEALTH SCIENCE CENTER AT FORT WORTH, Fort Worth, TX (US)

(72) Inventors: Jianye Ge, Fort Worth, TX (US); Bruce Budowle, Fort Worth, TX (US); Sammed Mandape, Fort Worth, TX (US); Jonathan King, Fort Worth, TX (US)

(21) Appl. No.: 18/263,914

(22) PCT Filed: Mar. 16, 2022
(86) PCT No.: PCT/US2022/020493

§ 371 Date: Aug. 2, 2023

Related U.S. Application Data
(60) Provisional application No. 63/161,568, filed on Mar. 16, 2021.

(51) Int. Cl. C12Q 1/6888

(52) U.S. Cl. CPC C12Q 1/6888; C12Q 2600/156; C12Q 2600/172

(57) ABSTRACT

The present invention includes a method for determining nucleic acid contributors to a sample from nucleic acids by determining one or more macrohaplotypes, comprising the steps of: obtaining or having obtained a sample; designing macrohaplotypes to obtain two or more markers selected from Short Tandem Repeat (STR), Single Nucleotide Polymorphisms (SNPs), Insertion-Deletions (Indels), or combinations thereof; generating amplicons or obtaining a sequence of amplicons from the sample from a paternal, maternal, or both chromosomes; sequencing the amplified products with LRS technologies; calling the haplotype variants of the sequence data; calculating from the one or more macrohaplotypes one or more nucleic acid contributors to the biological sample or specimen; comparing the one or more macrohaplotypes to a reference or known macrohaplotype profile from a subject suspected of contributing nucleic acids to the sample; and identifying a number of contributors to the sample.

### TECHNICAL FIELD OF THE INVENTION

The present invention relates in general to the field of forensic mixture evaluation, and more particularly, to the use of novel macrohaplotypes for forensic DNA mixture deconvolution.

### STATEMENT OF FEDERALLY FUNDED RESEARCH

Not applicable.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Mar. 16, 2022, is named UNTF2025WO_ST25.txt and is 7,873 bytes in size.

### BACKGROUND OF THE INVENTION

Without limiting the scope of the invention, its background is described in connection with deconvoluting crime scene DNA mixture samples.

Deconvoluting crime scene DNA mixture samples is one of the most challenging problems confronting forensic laboratories. This issue has been exacerbated given increased sensitivity of detection assays, increased emphasis on violent crime (e.g., sexual assault cases) and demand for analysis of high-volume crime (e.g., touch items with property crimes). Complex DNA mixture profiles with three or more contributors present particular challenges for analysts attempting to interpret profile(s), due to allele sharing, stochastic effects, etc. These challenges render some forensic evidence uninterpretable and thus cannot be used to develop investigative leads to solve the associated crimes.

The current forensic DNA markers for casework analyses primarily are based on the Short Tandem Repeat (STR) and on a more limited basis Single Nucleotide Polymorphisms (SNPs). These current marker systems (STRs, SNPs, Indels, or microhaplotypes) lack sufficient resolution to deconvolve mixture evidence.

Despite these advancements, a need remains for better mixture deconvolution that is compatible with existing technologies for sample preparation and forensic identification.

### SUMMARY OF THE INVENTION

In one embodiment, the present invention includes a method for determining nucleic acid contributors to a biological sample or specimen from nucleic acids obtained from single cells in the biological sample or specimen by determining one or more macrohaplotypes, comprising the steps of: obtaining or having obtained a biological sample or specimen; generating amplicons or obtaining a sequence of amplicons from the biological sample or specimen to obtain two or more markers selected from Short Tandem Repeat (STR), Single Nucleotide Polymorphisms (SNPs), Insertion-Deletions (Indels), or combinations thereof, from a paternal, maternal, or both chromosomes; calculating from the one or more macrohaplotypes one or more nucleic acid contributors to the biological sample or specimen; comparing the one or more macrohaplotypes to a reference or known macrohaplotype profile from a subject suspected of contributing nucleic acids to the biological sample or specimen; and identifying a number of contributors to the biological sample or specimen. In one aspect, the step of generating amplicons is by long-read sequencing. In another aspect, the amplicons are 100, 500, 1000, 2000, 3000, 4000, 5000, 6000, 7000, 8000, 10000, 50000, 100000 or more base pairs. In another aspect, the method further comprises determining one or more macrohaplotypes from the markers on the same paternal or maternal chromosome. In another aspect, the method further comprises comparing the one or more macrohaplotypes to a database of one or more nucleic acid-based forensic criminal databases and generating a list of investigative leads, an indictment document, or both. In another aspect, the macrohaplotype is further defined as a haplotype of a plurality of alleles determined from a plurality of markers on a paternal or a maternal chromosome. In another aspect, the macrohaplotype is further defined as a haplotype of a plurality of alleles determined from all the markers on a paternal or a maternal chromosome. In another aspect, the method further comprises determining using a probabilistic mixture model and the one or more processors, one or more genotypes of the one or more contributors at the one or more macrohaplotypes. In another aspect, the one or more nucleic acid contributors comprise 2, 3, 4, 5, 6, 7, 8, 9, 10 or more or more contributors. In another aspect, the biological sample or specimen comprises DNA molecules or RNA molecules. In another aspect, the biological sample or specimen comprises nucleic acid from zero, one, or more contaminant genomes and one genome of interest. In another aspect, the biological sample or specimen comprises cellular DNA. In another aspect, the one or more macrohaplotypes comprise at least one of the Short Tandem Repeat (STR), Single Nucleotide Polymorphisms (SNPs), and Insertion-Deletions (Indels). In another aspect, the macrohaplotype is sequenced using a forward and a reverse primer selected from SEQ ID NOS: 1 to 40.

In another embodiment, the present invention includes a method, implemented at a computer system that includes one or more processors and system memory, of quantifying a nucleic acid sample comprising nucleic acid of one or more contributors from one or more macrohaplotypes, the method comprising: obtaining or having obtained a biological sample or specimen; generating amplicons or obtaining a sequence of amplicons from the biological sample or specimen to obtain two or more markers selected from Short Tandem Repeat (STR), Single Nucleotide Polymorphisms (SNPs), Insertion-Deletions (Indels), or combinations thereof, from a paternal, maternal, or both chromosomes; calculating, with the one or more processors, from the one or more macrohaplotypes one or more nucleic acid contributors to the biological sample or specimen; comparing, with the one or more processors, the one or more macrohaplotypes to a reference or known macrohaplotype profile from a subject suspected of contributing nucleic acids to the biological sample or specimen; and identifying, with the one or more processors, one or more contributors to the biological sample by quantifying, using a probabilistic mixture model and the one or more processors, one or more fractions of nucleic acid of the one or more contributors in the nucleic acid sample, wherein using the probabilistic mixture model comprises deconvolution of nucleic acid mixtures from a complex mixture of two or more nucleic acid contributors. In one aspect, the method further comprises determining using a probabilistic mixture model and the one or more processors, one or more genotypes of the one or more contributors at the one or more macrohaplotypes. In another aspect, the one or more nucleic acid contributors comprise 2, 3, 4, 5, 6, 7, 8, 9, 10 or more or more contributors. In another aspect, the biological sample or specimen comprises DNA molecules or RNA molecules. In another aspect, the biological sample or specimen comprises nucleic acid from zero, one, or more contaminant genomes and one genome of interest. In another aspect, the biological sample or specimen comprises cellular DNA. In another aspect, the one or more macrohaplotypes comprise at least one of the Short Tandem Repeat (STR), Single Nucleotide Polymorphisms (SNPs), and Insertion-Deletions (Indels). In another aspect, the step of generating amplicons is by long-read sequencing. In another aspect, the amplicons are 100, 500, 1000, 2000, 3000, 4000, 5000, 6000, 7000, 8000, 10000, 50000, 100000 or more base pairs. In another aspect, the method further comprises determining one or more macrohaplotypes from the markers on the same paternal or maternal chromosome. In another aspect, the method further comprises comparing the one or more macrohaplotypes to a database of one or more nucleic acid-based forensic criminal databases and generating a list of investigative leads, an indictment document, or both. In another aspect, the macrohaplotype is further defined as a haplotype of a plurality of alleles determined from a plurality of markers on the same paternal or maternal chromosome. In another aspect, the macrohaplotype is further defined as a haplotype of a plurality of alleles determined from all the markers on the same paternal or maternal chromosome. In another aspect, the macrohaplotype is sequenced using a forward and a reverse primer selected from SEQ ID NOS: 1 to 40.

In another embodiment, the present invention includes a method for determining nucleic acid contributors to a biological sample or specimen from nucleic acids by determining one or more macrohaplotypes, comprising the steps of: obtaining or having obtained the biological sample or specimen; designing one or more macrohaplotypes to obtain two or more markers selected from Short Tandem Repeat (STR), Single Nucleotide Polymorphisms (SNPs), Insertion-Deletions (Indels), or combinations thereof; generating amplicons or obtaining a sequence of amplicons from the biological sample or specimen to obtain two or more markers selected from Short Tandem Repeat (STR), Single Nucleotide Polymorphisms (SNPs), Insertion-Deletions (Indels), or combinations thereof, from a paternal, maternal, or both chromosomes; sequencing the amplified products with long range sequencing (LRS) to obtain sequence data; calling haplotype variants from the sequence data; calculating from the one or more macrohaplotypes one or more nucleic acid contributors to the biological sample or specimen; comparing the one or more macrohaplotypes to a reference or known macrohaplotype profile from a subject suspected of contributing nucleic acids to the biological sample or specimen; and identifying a number of contributors to the biological sample or specimen.

In another embodiment, the present invention includes a method for generating sequences for one or more macrohaplotypes, comprising the steps of: (a) selecting one or more Short Tandem Repeat (STRs) (S) and a sequence length (L) of a predefined size; (b) determining one or more polymorphisms in the sequence surrounding S with a Single Nucleotide Polymorphisms (SNPs) and STR panel with n polymorphisms on a left side and m polymorphisms on the right size of S; (c) generating a list of possible macrohaplotypes with a size of L that contains S into a candidate list (Lₘ); (d) using a sliding window algorithm for all possible macrohaplotype configurations, wherein a window slides one polymorphism at a time from left to right, wherein a polymorphism sliding change creates a new macrohaplotype with one or more different polymorphism(s); (e) selecting the macrohaplotype with the lowest RMP on the candidate list (Lₘ); and repeating steps (a)-(e) for each STRs to generate a panel of optimal macrohaplotypes.

A kit for determining for determining nucleic acid contributors to a biological sample or specimen from nucleic acids by determining one or more macrohaplotypes, comprising: a container comprising one or more primer pairs for detecting macrohaplotypes from two or more markers selected from Short Tandem Repeat (STR), Single Nucleotide Polymorphisms (SNPs), Insertion-Deletions (Indels), or combinations thereof and reagents generating amplicons or obtaining a sequence of amplicons from the biological sample or specimen to obtain two or more markers selected from Short Tandem Repeat (STR), Single Nucleotide Polymorphisms (SNPs), Insertion-Deletions (Indels), or combinations thereof, from a paternal, maternal, or both chromosomes and for sequencing amplified products with long range sequencing (LRS) to obtain sequence data; instruction to: call haplotype variants from the sequence data; calculate from the one or more macrohaplotypes one or more nucleic acid contributors to the biological sample or specimen; compare the one or more macrohaplotypes to a reference or known macrohaplotype profile from a subject suspected of contributing nucleic acids to the biological sample or specimen; and identify a number of contributors to the biological sample or specimen. In one aspect, the amplicons are 100, 500, 1000, 2000, 3000, 4000, 5000, 6000, 7000, 8000, 10000, 50000, 100000 or more base pairs. In another aspect, the kit further comprises instructions for comparing the one or more macrohaplotypes to a database of one or more nucleic acid-based forensic criminal databases and generating a list of investigative leads, an indictment document, or both. In another aspect, the kit further comprises for determining using a probabilistic mixture model and the one or more processors, one or more genotypes of the one or more contributors at the one or more macrohaplotypes. In another aspect, the reagents amplify DNA molecules or RNA molecules. In another aspect, the macrohaplotype is sequenced using a forward and a reverse primer selected from SEQ ID NOS: 1 to 40.

### BRIEF DESCRIPTION OF THE DRAWINGS

For a more complete understanding of the features and advantages of the present invention, reference is now made to the detailed description of the invention along with the accompanying figures and in which:

FIG. 1 is a schema of a macrohaplotype, which includes one standard marker and variants in the flanking region (such as SNPs, Indels, STRs, etc.). In the macrohaplotype example of DNA sequence, A, T, C, and G are SNPs of the DNA sequences, (ATC)₂ and (GATA)₁₀ are STR markers, and “+” and “−” are the alleles of Indels.

FIGS. 2A and 2B show the distributions of observed distinct alleles (FIG. 2A) and Probability of Exclusion (FIG. 2B) for 2, 5, and 10 persons mixtures at D3S1358, vWA, and CSF1PO.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention uses a novel forensic marker system, the detection and/or determination of macrohaplotypes, which are large haplotypes that contain STRs and Single Nucleotide Variants (SNV) (including both SNPs and Indels), to significantly increase the number of alleles per marker determined and to improve mixture deconvolution. The present invention is compatible with existing STR data in national and local DNA databases. Thus, the macrohaplotype method disclosed herein enhances deconvolution of DNA mixtures better than existing marker systems.

As used herein, the term “amplification” refers to a method or reaction in which at least a part of at least one target nucleic acid is copied, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, any combinations thereof, such as, but not limited to: OLA/PCR, PCR/OLA, LDR/PCR, PCR/PCR/LDR, PCR/LDR, LCR/PCR, PCR/LCR, combined chain reaction (CCR), and the like. Descriptions of such techniques can be found in, among other sources, Ausbel et al.; PCR PRIMER: A LABORATORY MANUAL, Diffenbach, Ed., Cold Spring Harbor Press (1995); THE ELECTRONIC PROTOCOL BOOK, Chang Bioscience (2002); Msuih et al., J. Clin. Micro. 34:501-07 (1996); Innis et al., PCR PROTOCOLS: A GUIDE TO METHODS AND APPLICATIONS, Academic Press (1990), relevant portions incorporated herein by reference.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally. In other embodiments, amplification includes isothermal amplification methods. Isothermal amplification uses a constant temperature rather than cycling through denaturation and annealing/extension steps. Some means of strand separation, e.g., an enzyme, is used in place of thermal denaturation.

For use with the present invention, amplicons can be produced upon preamplification and/or amplification, that are conveniently analyzed by an amplification method, such as PCR. In particular embodiments, as amplified sample from a single cell or small cell population may be used for many separate PCR reactions performed in a low-volume PCR reaction apparatus. In certain embodiments, preamplification is carried out using one or more primer pairs specific for the one or more target nucleic acids of interest. Thus, a low-volume PCR reaction apparatus can include separate reaction chambers for amplifying with each primer pair, such that the production of an amplicon in a particular reaction chamber indicates that the corresponding target nucleic acid was present in the sample.

Detection of amplicons is carried out using methods known in the art. These can include fluorometric methods, such as real-time quantitation method that monitoring the formation of amplification product involves the continuous measurement of PCR product accumulation using a dual-labeled fluorogenic oligonucleotide probe, e.g., a TaqMan® and U.S. Pat. No. 5,723,591, relevant portions incorporated herein by reference. TaqMan® is widely used for qPCR and the present invention is not limited to use of TaqMan® probes, but also, any suitable probes can be used with the present invention.

As used herein, the terms “biological sample” or “biological specimen” refers a biological fluid, tissue, residue or surface on which single cells or portions thereof can be obtained and are from a biological source. The samples or specimens are obtained and prepared using conventional methods known in the art. In particular, DNA or RNA are useful in the methods described herein and can be extracted and/or amplified from any source. Suitable nucleic acids can also be obtained from an environmental source (e.g., water), from man-made products (e.g., food), from forensic samples, and the like. Nucleic acids can be extracted or amplified from cells or portions thereof, bodily fluids (e.g., blood, a blood fraction, urine, feces, bodily secretions, etc.), or tissue samples by any of a variety of standard techniques. Non-limiting examples of samples or specimens include skin surfaces, genital areas or tracts, rectum, plasma, serum, spinal fluid, lymph fluid, peritoneal fluid, pleural fluid, oral fluid, samples from the respiratory, intestinal, genital, and urinary tracts; samples of tears, saliva, blood cells, from textiles (such as bedding or carpet), from door handles, etc. Samples can be obtained from live or dead organisms or processed products of organisms. Illustrative samples can include single cells, paraffin-embedded tissue samples, needle biopsies, and food products. Nucleic acids useful in the methods described herein can also be derived from one or more nucleic acid libraries, including cDNA, cosmid, YAC, BAC, P1, PAC libraries, and the like.

Nucleic acids of interest can be isolated using methods well known in the art, with the choice of a specific method depending on the source, the nature of biological sample or specimen, the nucleic acid, and environmental factors. The sample nucleic acids need not be in pure form but are typically sufficiently pure to allow the amplification steps of the methods described herein to be performed.

Where the target nucleic acids are mRNA, the RNA can be reversed transcribed into cDNA by standard methods known in the art and as described in Sambrook, J., Fritsch, E. F., and Maniatis, T., Molecular Cloning: A Laboratory Manual. Cold Spring Harbor Laboratory Press, NY, Vol. 1, 2, 3 (1989), relevant portions incorporated herein by reference. cDNA can be analyzed according to the methods described herein.

As used herein, the term “hybridization” refers to the binding of a nucleic acid to a target nucleotide sequence in the absence of substantial binding to other nucleotide sequences present in the hybridization mixture under defined stringency conditions, such as low, medium, or high stringency.

Those of skill in the art recognize that relaxing the stringency of the hybridization conditions allows sequence mismatches to be tolerated. In particular embodiments, hybridizations are carried out under stringent hybridization conditions as taught in, e.g., Berger and Kimmel (1987) METHODS IN ENZYMOLOGY, VOL. 152: GUIDE TO MOLECULAR CLONING TECHNIQUES, San Diego: Academic Press, Inc. and Sambrook et al. (1989) MOLECULAR CLONING: A LABORATORY MANUAL, 2ND ED., VOLS. 1-3, Cold Spring Harbor Laboratory), relevant portions incorporated herein by reference). The melting temperature of a hybrid (and thus the conditions for stringent hybridization) is affected by various factors such as the length and nature (DNA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Illustrative stringent conditions suitable for achieving specific hybridization of most sequences are: a temperature of, e.g., at least about 65 degrees C. and a salt concentration of, e.g., 0.2 molar at pH7.

As used herein, the term “nucleic acid” refers to polynucleotides including natural nucleotides and nucleotide analogs that can function (e.g., hybridize) in a similar manner to naturally occurring nucleotides. The term nucleic acid includes any form of DNA or RNA, including, for example, genomic DNA; complementary DNA (cDNA), mRNA, other RNAs, DNA molecules produced synthetically or by amplification. The term nucleic acid also includes any chemical modification of the polynucleotides, such as by methylation and/or by capping. Nucleic acid modifications can include, e.g., chemical groups that incorporate additional charges, polarizability, hydrogen bonding, electrostatic interaction, and functionality to the individual nucleic acid bases, phosphodiester bonds, or to the nucleic acid as a whole. Nucleic acid(s) can be obtained a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “nucleotide tag” refers to a predetermined nucleotide sequence that is added to a target nucleotide sequence. The nucleotide tag can encode an item of information about the target nucleotide sequence, such the identity of the target nucleotide sequence, the chromosome from which that sequence derives, or the identity of the sample from which the target nucleotide sequence was derived. Nucleotide tag sequences are generally not used as primer binding sites in the first round of amplification.

As used herein, the term “oligonucleotide” refers to a polynucleotide that is relatively short, generally in the 15-25 range, but generally in the 20-30, 30-40, 40-50, 80, 90, 100, 125, 150, 175 or 200 nucleotide range. Typically, oligonucleotides are single-stranded DNA molecules, but double-stranded oligonucleotides can also be produced.

As used herein, the terms “polymorphic marker” or “polymorphic site” refer to a locus at which nucleotide sequence variance occurs. Illustrative markers have at least two alleles, each occurring at frequency of greater than 1% (lower percentages also are considered polymorphic), and more typically greater than 1% of a selected population. A polymorphic site can be as small as one base pair. Polymorphic markers include restriction fragment length polymorphism (RFLPs), variable number of tandem repeats (VNTR's), hypervariable regions, minisatellites, dinucleotide repeats, trinucleotide repeats, tetranucleotide repeats, pentanucleotide repeats, hexanucleotide repeats and beyond, simple sequence repeats, deletions, and insertion elements such as Alu. The first identified allelic form is arbitrarily designated as the reference form and other allelic forms are designated as alternative or variant alleles. The allelic form occurring most frequently in a selected population may sometimes be referred to as the wildtype form. Diploid organisms can be homozygous or heterozygous for allelic forms. A diallelic polymorphism has two forms, while a triallelic polymorphism has three forms, et seq.

As used herein, the term “primer” refers to an oligonucleotide that is capable of hybridizing or annealing with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but primers are typically at least 6 nucleotides long and, more typically range from 10 to 30 nucleotides, or even more typically from 15 to 30 nucleotides, in length. Other primers can be somewhat longer, e.g., 30 to 50 nucleotides long. In this context, “primer length” refers to the length of an oligonucleotide or nucleic acid that hybridizes to a complementary “target” sequence and primes nucleotide synthesis. Short primer molecules generally require cooler temperatures to form sufficiently stable hybrid complexes with the template. A primer need not reflect the exact sequence of the template but must be sufficiently complementary to hybridize with a template.

As used herein, the term “primer site” or “primer binding site” refers to a segment of a target nucleic acid to which a primer hybridizes. A primer can include a nucleotide tag, e.g., appended to its 5′ end.

A primer is said to anneal to another nucleic acid if the primer, or a portion thereof, specifically hybridizes to a nucleotide sequence within the nucleic acid. The statement that a primer hybridizes to a particular nucleotide sequence is not intended to imply that the primer hybridizes either completely or exclusively to that nucleotide sequence.

As used herein, the term “primer pair” refers to a set of primers including a 5′-“upstream primer” or “forward primer” that hybridizes with the complement of the 5′-end of the DNA sequence to be amplified and a 3′-downstream primer (or reverse primer) that hybridizes with the 3′ end of the sequence to be amplified. As will be recognized by those of skill in the art, the terms “upstream” and “downstream” or “forward” and “reverse” are not intended to be limiting, but rather provide illustrative orientation in particular embodiments. A primer pair is said to be “unique” if it can be employed to specifically amplify a particular target nucleotide sequence in a given amplification mixture.

As used herein, the term “probe” refers to a nucleic acid capable of binding to a target nucleic acid of complementary sequence through one or more types of chemical bonds, generally through complementary base pairing, usually through hydrogen bond formation, thus forming a duplex structure. The probe binds or hybridizes to a “probe binding site.” The probe can be labeled with a detectable label to permit facile detection of the probe, particularly once the probe has hybridized to its complementary target. Alternatively, however, the probe can be unlabeled, but can be detectable by specific binding with a ligand that is labeled, either directly or indirectly. Probes can vary significantly in size. Generally, probes are at least 6 to 15 nucleotides in length. Other probes are at least 10, 15, 20, 25, 30, or 40 nucleotides long. Still other probes are somewhat longer, being at least 50, 60, 70, 80, or 90 nucleotides long. Yet other probes are longer still and are at least 100, 150, 200 or more nucleotides long. Probes can also be of any length that is within any range bounded by any of the above values (e.g., 15-20 nucleotides in length). Primers can also function as probes. Typically, the primer or probe can be perfectly complementary to the target nucleic acid sequence or can be less than perfectly complementary. In certain embodiments, the primer has at least 65% identity to the complement of the target nucleic acid sequence over a sequence of at least 7 nucleotides, more typically over a sequence in the range of 10-30 nucleotides, and often over a sequence of at least 14-25 nucleotides, and more often has at least 75% identity, at least 85% identity, at least 90% identity, or at least 95%, 96%, 97%. 98%, or 99% identity. It will be understood that certain bases (e.g., the 3′ base of a primer) are generally complementary to corresponding bases of the target nucleic acid sequence. Primer and probes typically anneal most specifically to the target sequence under stringent hybridization conditions.

As used herein, the term “qPCR” refers to quantitative real-time polymerase chain reaction (PCR), which is also known as “real-time PCR” or “kinetic polymerase chain reaction.”

As used herein, the term “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, nucleotides, labels, dyes, nucleases, and the like. Reagents for enzyme reactions include, for example, enzymes, substrates, cofactors, buffer, metal ions, inhibitors, and activators.

As used herein, the term “single nucleotide polymorphism” (SNP) refers to a polymorphic site occupied by a single nucleotide (although the nucleotides can be any number within a group), which is the site of variation between allelic sequences. The site is usually preceded and followed by highly conserved sequences of the allele (e.g., sequences that vary in less than 1/100 or 1/1000 members of the populations). A SNP usually arises due to substitution of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele. In certain embodiments, a collection of SNPs, mRNAs, non-coding RNAs (e.g., miRNAs), etc., can be identified are used to determine the one or more nucleic acid contributors to a biological sample or specimen from nucleic acids obtained from single cells.

As used herein, the term “target-specific qPCR probe” refers to a qPCR probe that identifies the presence of an amplification product during qPCR, based on hybridization of the qPCR probe to a target nucleotide sequence present in the product.

Target nucleic acids can be amplified and can be detected using the methods described herein. In some embodiments, at least some nucleotide sequence will be known for the target nucleic acids. For example, if PCR is used for preamplification/amplification of target nucleic acids, sufficient sequence information is typically available for each end of a given target nucleic acid to permit design of suitable amplification primers, although, those of skill in the art appreciate that target nucleic acids of unknown sequence can be amplified (e.g., using a pool of degenerate primers or a pool of combinatorial primers, such as random hexamers) as can mRNA (e.g., using oligo-dT). Target nucleic acids include polymorphisms, such as single nucleotide polymorphisms (SNPs). In this case, the amplification primers can be SNP-specific, meaning that at least one primer hybridizes to a SNP, such that an amplicon is produced only if the SNP is present and quantity in the sample nucleic acids.

Typical thermal cycling devices and reactions can be used with the present invention such a fluorescent dyes that emit a light beam of a specified wavelength, and detectors that read the intensity of the fluorescent dye. Devices for use with the present invention include, but are not limited to devices that can include one or more of the following: a thermal cycler, light beam emitter, and a fluorescent signal detector, have been described, e.g., in U.S. Pat. Nos. 5,928,907; 6,015,674; and 6,174,670, relevant portions incorporated herein by reference. Thermal cycling and fluorescence detecting devices can be used for precise quantification of target nucleic acids. In some embodiments, fluorescent signals can be detected and displayed during and/or after one or more thermal cycles, thus permitting monitoring of amplification products as the reactions occur in “real-time.” In certain embodiments, one can use the amount of amplification product and number of amplification cycles to calculate how much of the target nucleic acid sequence was in the sample prior to amplification.

According to some embodiments, amplification products are monitored after a predetermined number of cycles to indicate the presence of the target nucleic acid sequence in the sample. One skilled in the art can easily determine, for any given sample type, primer sequence, and reaction condition, how many cycles are sufficient to determine the presence of a given target nucleic acid.

As used herein, the term “target nucleic acids” refers to specific nucleic acids to be detected, such as Short Tandem Repeat (STR), Single Nucleotide Polymorphisms (SNPs), Insertion-Deletions (Indels), sequences adjacent thereto, and the like. Target nucleic acids include, for example, loci of interest (STRs, SNPs, Indels). Target nucleic acids can also be RNA or DNA.

Non-coding RNAs include those RNA species that are not necessarily translated into protein. These include, but are not limited to, transfer RNA (tRNA) and ribosomal RNA (rRNA), as well as RNAs such as small nucleolar RNAs, microRNAs, small interfering RNAs, Piwi-interacting RNAs (piRNAs, particularly those in spermatogenesis), and long non-coding RNAs (long ncRNAs.

As used herein, the term “target nucleotide sequence” refers to a molecule that has the nucleotide sequence of a target nucleic acid, e.g., an amplification product obtained by amplifying a target nucleic acid or the cDNA produced upon reverse transcription of an mRNA target nucleic acid.

As used herein, the term a “complementary sequence” refers to polynucleotides with the capacity for binding between two nucleotides, e.g., a nucleotide at a given position is capable of hydrogen bonding with a nucleotide of another nucleic acid, then the two nucleic acids are considered to be complementary to one another at that position. As used herein, complementarity refers to traditional Watson-Crick or non-canonical pairing between two single-stranded nucleic acid molecules can be partial, in which only some of the nucleotides bind, or it can be complete complementarity when total sequence alignment exists between the single-stranded molecules. The degree of complementarity between nucleic acid strands has significant effects on the efficiency and strength of hybridization between nucleic acid strands and the consequent stacking interactions.

As used herein, the term “universal detection probe” refers to any probe that identifies the presence of an amplification product, regardless of the identity of the target nucleotide sequence present in the product.

As used herein, the term “universal qPCR probe” refers to any such probe that identifies the presence of an amplification product during qPCR. In certain embodiments, one or more amplification primers can comprise a nucleotide sequence to which a detection probe, such as a universal qPCR probe binds. In this manner, one, two, or more probe binding sites can be added to an amplification product during the amplification step of the methods described herein. Those of skill in the art recognize that the possibility of introducing multiple probe binding sites during preamplification (if carried out) and amplification facilitates multiplex detection, wherein two or more different amplification products can be detected in a given amplification mixture or aliquot thereof.

As used herein, the term “universal detection probe” refers to primers labeled with a detectable label (e.g., a fluorescent label), as well as non-sequence-specific probes, such as DNA binding dyes, including double-stranded DNA (dsDNA) dyes, such as SYBR Green.

The present invention uses a novel forensic marker system, macrohaplotypes, which are large haplotypes that contain STRs, SNPs, and Indels that can significantly increase the number of alleles per marker and improve mixture deconvolution. The present invention can work with existing systems, including backward compatibility with STR data in national and local DNA databases. The macrohaplotype enhances deconvolution of DNA mixtures better than with existing marker systems.

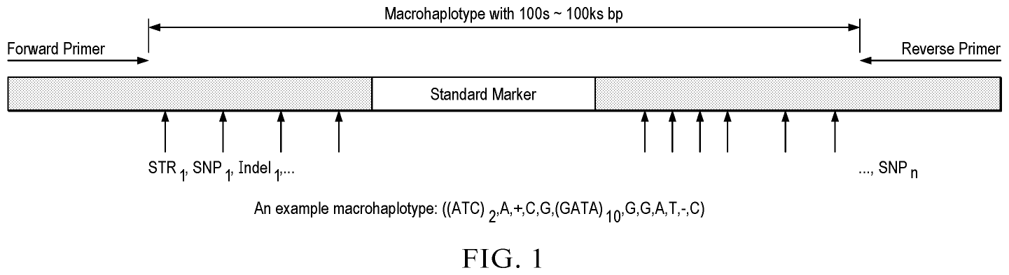

The novel forensic marker system of the present invention detects macrohaplotypes, which combine CODIS STR and flanking variants in an extended fashion. FIG. 1 shows the general design of a macrohaplotype, in which a forensically-relevant, or for that matter any, STR is resident in a macrohaplotype, together with SNPs, Insertion-Deletions (Indels), and other STRs in the flanking region(s). A macrohaplotype is a haplotype with the alleles of all markers on the same paternal or maternal chromosome. The macrohaplotypes markedly increase the number of alleles (or haplotypes) compared with any other component markers contained within the fragment (namely, from ˜10s to ˜100s or even ˜1,000s), and significantly increase the statistical strength on a per marker basis, which can be used to better serve forensic applications. Particularly for mixture deconvolution purpose, the macrohaplotypes substantially reduce the chance of observing allele overlap among different contributors compared with current capabilities based on STR markers. Therefore, macrohaplotypes provide higher accuracies to determine the number of contributors of a mixture, particularly with complex mixtures ≥3 contributors, and offer much higher probabilities to exclude non-contributors. Macrohaplotypes are compatible with fundamental interpretation and statistical methods such as likelihood ratio-based mixture interpretation methods. In addition, the macrohaplotypes are compatible with current forensic databases, since they can be constructed to contain forensically relevant STRs, such as Combined DNA Index System (CODIS) core STRs. Thus, the STR genotypes derived from deconvolved macrohaplotypes could be uploaded to CODIS for searching for matching profiles to yield strong investigative leads and even generate indictment documents or evidence.

Example of Data sources. Saini et al. [37] created a genome-wide SNP+STR haplotype reference panel based on the Simons Simplex Collection Phase 1 dataset and the 1000 Genomes Project Phase 3 data (https://gymreklab.github.io/2018/03/05/snpstr_imputation.html). The genome coordinate of the Saini's SNP+STR panel was GRCh37 (Genome Reference Consortium Human Build 37), and it could be converted to GRCh38 with an online tool LiftOver (http://genome.ucsc.edu/cgi-bin/hgLiftOver). In addition, the 1000 genomes data [38] with GRCh38 provide much more phased SNVs than those of GRCh37 (ftp://ftp.1000genomes.ebi.ac.uk/voll/ftp/release/20130502/supporting/GRCh38_positions/). Therefore, the Saini's panel was updated by merging this panel and the GRCh38 version of the 1000 genomes data to include more variants. In addition, Phillips et al. [39] compiled the coordinates of CODIS STRs based on the GRCh38 coordinate. Combining all these data together created an updated SNP+STR haplotype panel in GRCH38, including 2,504 unrelated samples from 26 populations in 5 super populations, which served as foundations of designing macrohaplotypes.

During the merging of the Saini's panel and the GRCh38 version of the 1000 genomes data, the same phases were kept by comparing the overlapped SNVs in both datasets. The SNVs in the 1000 genomes data that overlapped with the CODIS STR regions were removed to avoid double-counting of the polymorphisms. The Saini's panel identified many homopolymer regions as STRs. These homopolymers were excluded from the updated panel, since the homopolymers are more likely to include sequence errors.

The sequences of two CODIS STRs, D16S539 and D21S11, were not captured in the Saini's panel, thus they were not included in the updated SNP+STR panel. However, the genomic coordinates of these two STRs were available and used in designing the optimal macrohaplotypes as described below.

The size of a macrohaplotype is only limited by the sequencing technologies and the size of intact DNA in a sample. Thus, a single macrohaplotype with multiple million base pairs in length could alone deconvolute a complex mixture. In reality, based on the current widely used technologies for genomic DNA extraction, sample preparation, library preparation, and long-read sequencing, as well as the condition of common forensic mixture samples, macrohaplotypes with sizes of 8˜10 k bp achieve good quality sequencing results with sufficient read depth for forensic type samples. However, the present invention includes both shorter and longer sequences sufficient to generate the macrohaplotype and allow for deconvolution of mixtures. As used herein, the phrase “long range sequencing” involves determining a sequence of 0.5, 1, 2, 3, 4, 5, 6, 7, 8, 9, 10, 12, 15, 20, 30, 40, 50, 100, 150, 200, 250, 300, 400, 500, 600, 700, 750, 800, or 900 kilobases, but can also include, 1, 2, 3, 4, 5, 6, 7, 8, 9, 10, 12, 15, 20, 30, 40, 50, 100, 150, 200, 250, 300, 400, 500, 600, 700, 750, 800, or 900 megabases,

Example 1. Macrohaplotype design process. Based on the updated SNP+STR haplotype panel with GRCh38, one macrohaplotype can be designed for each given CODIS STR with a predefined length. Thus, 20 macrohaplotypes were designed, and each anchored on a CODIS STR. In this study, a size of 8 k bp was used for illustrative purposes, because long DNA sequences of RFLP have been successfully amplified and sequenced with forensic samples [28]. In real casework, an appropriate macrohaplotype size can be determined by evaluating the quality of the samples (i.e., DNA fragment sizes), the requirement of discrimination power for the particular application(s), available sequencing technologies, etc. It should be noted that while 8 kb was the length selected for this study, smaller size macrophaplotypes (e.g., 1-7 kb) can also be designed that still would provide extremely high discrimination power. The size can also be 500 bp or 10,000+bp. The size of the microhaplotype (from a panel of validated varied length macrohaplotypes) can be selected depending on a quality assessment of a sample. The 8 kb length in the study herein was selected for this example.

With one example of a size of the macrohaplotypes decided, a sliding window algorithm was used to look for the start and end positions (or polymorphisms) of the macrohaplotypes with the maximum discrimination power or the lowest Random Match Probability (RMP). The algorithm details are described as follows.
a. Let S be a CODIS STR and L be the predefined size (i.e., sequence length) of the macrohaplotype for the STR (e.g., 8 k bp).
b. Determine all polymorphisms in the sequence surrounding S with the SNP+STR panel as there are n polymorphisms on the left side of the SM and m polymorphisms on the right size of S, i.e., (P₁₁, P₁₂, . . . , Pₗₙ, S, Pᵣ₁, Pᵣ₂, . . . , Pᵣₘ), where P₁₁, P₁₂, . . . , Pₗₙ, are the polymorphisms on the left side of S and Pᵣ₁, Pᵣ₂, . . . , Pᵣₘ are the polymorphisms on the right size of S.
c. List all possible macrohaplotypes with a size of L that contains S into a candidate list (Lₘ). Namely, a macrohaplotype includes all polymorphisms of (Pₗᵢ, . . . , Pₗₙ, S, Pᵣ₁, . . . , Pᵣⱼ) with of a size of L, in which Pₗᵢ, and Pᵣⱼ are the leftmost and the rightmost polymorphisms, respectively. A macrohaplotype may start with S, such as (S, Pᵣ₁, . . . , Pᵣⱼ), or end with S, such as (Pₗᵢ, . . . , Pₗₙ, S). Using a sliding window algorithm, all possible macrohaplotype configurations, with start positions and included polymorphisms defined, can be listed on Lₘ. The window slides one polymorphism at a time from left to right. One polymorphism sliding change will create a new macrohaplotype with some different polymorphism(s) included and/or excluded.
d. Select the macrohaplotype with the lowest RMP on the candidate list (Lₘ) as the optimal macrohaplotype associated with S·RMP=Σᵢ(Gᵢ)², in which Gᵢ is the genotype frequency of the i-th genotype of this macrohaplotype.
e. Repeat steps a-d for each CODIS STR to generate a panel of 20 optimal macrohaplotypes.

Statistical evaluation of macrohaplotypes. The RMP and effective number of alleles (Ae) were calculated for the designed macrohaplotypes based on the updated SNP+STR GRCh38 panel. Ae is the reciprocal of the homozygosity, Ae=1/Σᵢ(fᵢ)² where fᵢ is the frequency of the i-th allele (or haplotype) [40].

Arlequin software was used to calculate the population substructure Fst value between and among populations, test the departure from Hardy-Weinberg Equilibrium (HWE) for each macrohaplotype, and test LD between each macrohaplotype pair. Arlequin is a free population genetics software that performs several types of tests and calculations, including Fixation index (Fst, also known as the “F-statistics” [2]), computing genetic distance, Hardy-Weinberg equilibrium, linkage disequilibrium, mismatch distribution, and pairwise difference tests.

In addition, to evaluate the capabilities of the macrohaplotypes for mixture interpretation, DNA mixtures with 2 to 10 contributors were randomly simulated for each macrohaplotypes without considering mixture ratios or read depths. For each simulated mixture, the number of the observed distinct alleles (or haplotypes) and the probability of exclusion (PE) were calculated. PE=1−(Σᵢfᵢ)², where fᵢ is the frequency of the i-th allele (or haplotype) observed in the mixture profile. The distributions of the number of distinct alleles and PE were plotted by ggplot2 (v. 3.3.3) in R [Wickham H. (2016) ggplot2: elegant graphics for data analysis. Springer].

Following the design process described above, 20 macrohaplotype were generated (Table 1), and the haplotypes of these macrohaplotypes were extracted from the updated SNP+STR GRCh38 panel. Each haplotype of the macrohaplotypes (or macrohaplotype's allele) is a series of the combined variants included in the macrohaplotypes, as illustrated in FIG. 1. On average, there were 771 haplotypes for each macrohaplotypes across all the populations, compared with 21 alleles per CODIS STR based on the Saini's panel. The names and positions of the variants within the macrohaplotypes. The first and the last variants, together with their physical positions, also can be found in Table 1. On average, there were 264 variants in one macrohaplotype, with a minimum of 202 at FGA and a maximum of 448 at D16S539. Non-CODIS STRs were observed in almost all of these macrohaplotypes, except with D16S539. The macrohaplotype of D1S1656 had 3 non-CODIS STRs. The CODIS STR sequences of D21S11 and D16S539 were not captured in the Saini's panel, and thus the two corresponding macrohaplotypes did not include the CODIS STR variants. If CODIS STRs were included in these two macrohaplotypes, the discrimination powers for these loci should be higher.

TABLE 1
The optimal macrohaplotypes, based on random match probability (RMP), with sizes of 8k bp. Start_var and End_var are
the first and last variants in a macrohaplotype, respectively. Start_Pos and End_pos are the physical positions (GRCh38) of
Start_var and End_var, respectively. Ae is the effective number of alleles. Fst was calculated across all 26 populations.

| | | | | | | No. | No. | | | RMP | Ae | |
|---|---|---|---|---|---|---|---|---|---|---|---|---|
| | Macro- | | | | | of | of | | | (CODIS | (CODIS | |
| Chr | haplotype | Start_var | End_var | Start_Pos | End_Pos | variants | STR | RMP | Ae | STR only) | STR only) | Fst |
| 1 | D1S1656 | rs552031105 | rs551695968 | 230764792 | 230772773 | 273 | 4 | 6.42E−05 | 174.8 | 1.61E−02 | 10.5 | 0.0098 |
| 2 | TPOX | rs187386486 | rs11404899 | 1484164 | 1492164 | 339 | 2 | 1.15E−03 | 36.7 | 1.51E−01 | 3.1 | 0.0218 |
| 2 | D2S441 | rs376229298 | rs542126907 | 68007898 | 68015891 | 259 | 3 | 8.95E−04 | 43.2 | 6.98E−02 | 4.8 | 0.0170 |
| 2 | D2S1338 | rs576468026 | rs570093841 | 218010354 | 218018340 | 247 | 1 | 1.93E−04 | 99.5 | 1.12E−02 | 12.8 | 0.0156 |
| 3 | D3S1358 | rs555060850 | rs111807780 | 45533828 | 45541806 | 210 | 3 | 3.69E−03 | 20.9 | 2.86E−02 | 7.7 | 0.0417 |
| 4 | FGA | rs532470007 | rs144037270 | 154587666 | 154595656 | 202 | 2 | 7.74E−04 | 49.8 | 3.14E−02 | 7.6 | 0.0217 |
| 5 | D5S818 | rs574978603 | rs551775352 | 123768746 | 123776743 | 251 | 1 | 2.01E−03 | 30.5 | 6.80E−02 | 4.8 | 0.0346 |
| 5 | CSF1PO | CSF1PO | rs10043508 | 150076321 | 150084310 | 272 | 2 | 5.61E−05 | 185.4 | 1.18E−01 | 3.7 | 0.0114 |
| 7 | D7S820 | rs74937330 | rs531702847 | 84152889 | 84160837 | 216 | 1 | 7.92E−04 | 49.0 | 8.24E−02 | 4.5 | 0.0177 |
| 8 | D8S1179 | rs76501817 | rs576029037 | 124888113 | 124896109 | 255 | 1 | 2.92E−04 | 81.1 | 2.15E−02 | 9.1 | 0.0128 |
| 10 | D10S1248 | rs553906439 | rs577805463 | 129286446 | 129294445 | 234 | 1 | 6.10E−03 | 17.0 | 8.36E−02 | 4.5 | 0.0357 |
| 11 | TH01 | rs567980491 | rs574796022 | 2170936 | 2178931 | 347 | 2 | 2.70E−03 | 25.6 | 7.40E−02 | 4.8 | 0.0363 |
| 12 | vWA | rs139605275 | rs540190270 | 5983521 | 5991499 | 272 | 2 | 5.56E−04 | 58.7 | 4.69E−02 | 6.0 | 0.0192 |
| 12 | D12S391 | D12S391 | rs528853599 | 12297020 | 12304996 | 279 | 2 | 2.94E−04 | 79.8 | 1.12E−02 | 12.2 | 0.0148 |
| 13 | D13S317 | rs536343928 | rs561009956 | 82140525 | 82148520 | 202 | 2 | 1.52E−03 | 33.4 | 3.29E−02 | 7.4 | 0.0305 |
| 16 | D16S539* | rs549533871 | rs572690281 | 86346988 | 86354985 | 448 | 0 | 9.62E−04 | 42.7 | | | 0.0376 |
| 18 | D18S51 | rs182811931 | rs558695830 | 63275718 | 63283702 | 225 | 1 | 1.99E−04 | 97.3 | 2.70E−02 | 8.2 | 0.0106 |
| 19 | D19S433 | rs546251734 | rs138538752 | 29926187 | 29934178 | 249 | 2 | 4.57E−05 | 207.0 | 5.11E−02 | 5.7 | 0.0058 |
| 21 | D21S11* | rs559045572 | rs529204722 | 19179141 | 19187083 | 210 | 1 | 1.63E−03 | 33.2 | | | 0.0188 |
| 22 | D22S1045 | rs190864081 | rs148319179 | 37140272 | 37148250 | 285 | 2 | 1.52E−04 | 109.5 | 9.35E−02 | 4.2 | 0.0104 |

*The sequences of CODIS STRs D21S11 and D16S539 were not captured in the Saini's panel [37].

Given the genomic (hg38) coordinates of the proposed macrohaplotypes (Table 1), the DNA sequence of the reference human genome plus up to 400 bp of flanking region beyond both the start and end marker was extracted from reference genome using the bedtools function getfasta. This sequence then was used to generate potential primers with the online application Primer3 (Table 2). Potential primers then were validated using UCSC's In-Silico PCR online application for specificity to the targeted region.

TABLE 2
Examples of Designed forward primers and reverse primers for the macrohaplotypes
in Table 1.

| | Macro- | | SEQ | | SEQ |
|---|---|---|---|---|---|
| Chr | haplotype | Forward primers | ID NO | Reverse primers | ID NO |
| 5 | CSF1PO | TGCACACTTGGACAGCATTT | 1 | GACTCCATCTCCTTCCTTTCTT | 2 |
| 10 | D10S1248 | AAACTGATGCTCTTCAAAGGC | 3 | AGTGGTTGTCTTAGCTTGCA | 4 |
| 12 | D12S391 | CCAGAGAGAAAGAATCAACAGGA | 5 | AGATCTCTTCCTCCAAACTGCA | 6 |
| 13 | D13S317 | AGGGACATGGATGAAGCTGG | 7 | TGAGTAAGTCATAGAGGAGGTCG | 8 |
| 16 | D16S539 | AAGCTCAGAGAGGGGAACTG | 9 | AGTGCTTCCCCTGCTCAATA | 10 |
| 18 | D18S51 | CATTTTGAGAGTGCCCCGAG | 11 | AAGAGGCCCTGGTGACTTAG | 12 |
| 19 | D19S433 | GGTGAACAAAAGGACCTTGGA | 13 | AGCAATTTGTGAGGCCAAGG | 14 |
| 1 | D1S1656 | GAGTGAACGGATGGTGGATG | 15 | GGGGACACACACAGAAAAGG | 16 |
| 21 | D21S11 | GCAAATGGGCAATTGAGTGT | 17 | TCCAGCCTACATCCACATCT | 18 |
| 22 | D22S1045 | GACCCTGTCCTAGCCTTCTT | 19 | AGCCTCAGTGACTGCCAG | 20 |
| 2 | D2S1338 | CAGAGTTCCGGGGTTGGG | 21 | GGCCAGCCTGTTTTCTTGC | 22 |
| 2 | D2S441 | CAGATCACGAGGTCAGGAGT | 23 | GTGGCCAGAACTTCCAACAC | 24 |
| 3 | D3S1358 | ACCAGATCTCCAACAGGACA | 25 | GAGCTTCCTCGGCACCAG | 26 |
| 5 | D5S818 | CTAGCTTGCCATTCTGTGCC | 27 | ACCTTAGAACACACCCAATTCA | 28 |
| 7 | D7S820 | GGCTGTGTCTCTAAGTGGCA | 29 | AGTTTCACTCTTGTTGCCCA | 30 |
| 8 | D8S1179 | GCAGCACCATCTTTCACAGT | 31 | AAGGAAGGAGAGAGGTAGCA | 32 |
| 4 | FGA | ATGACTTTGCGCTTCAGGAC | 33 | AGCTTTGCCAATGTTGTCCA | 34 |
| 11 | TH01 | GGTACCTGGAAATGACACTGC | 35 | TGATTGAGTCACCGGCATG | 36 |
| 2 | TPOX | TCGTAATTTCCAGGCCCTGT | 37 | AGATCACCCCATTGCTCTCC | 38 |
| 12 | vWA | GGAGACAGAGATTACATGGGTT | 39 | TGGTTCAAATCCTGCGTCTG | 40 |

With so many variants included in the macrohaplotypes, the discrimination powers were significantly increased compared with the CODIS STRs alone. The average Ae value of the macrohaplotypes was 73.8, with a maximum of 207 at D19S433 and a minimum of 17 at D10S1248. In contrast, the average Ae of the CODIS STRs was only 6.8, which was about 9-times lower than that of the macrohaplotypes. The geometric mean of the RMPs of all 20 macrohaplotypes was 5.58×10⁻⁴, with a maximum of 6.10×10⁻³ at D10S1248 (the lowest discrimination power) and a minimum of 4.57×10⁻⁵ at D19S433 (the highest discrimination power). In contrast, the geometric mean of the RMPs of the length-based CODIS STRs (called in Saini's panel) was only 4.37×10⁻², which was about two magnitudes less informative than that of the macrohaplotypes. The highest and lowest RMP differences between the CODIS STRs and the associated macrohaplotypes were found at CSF1PO and D3S1358 (i.e., 2,103.4 and 7.8 times different), respectively, with a geometric mean difference of 84.4. On average, one macrohaplotype is equivalent to 2˜3 CODIS STRs, in terms of RMP.

The average Fst value of the macrohaplotypes among all the populations was 0.021, with a maximum of 0.0417 at D3S1358 and a minimum of 0.0058 at D19S433. The pairwise Fst between each pair of populations based on the macrohaplotypes. The Fst value of each super population is expected to be lower, since the variations of the haplotype frequencies within each super population are expected be smaller. Further, a Multidimensional scaling (MDA) was plotted by R [42] to visualize the distance between the populations. In general, these macrohaplotypes were able to clearly differentiate the African, East Asian, and South Asian. As expected, some Admixed American populations (i.e., CLM: Colombians from Medellin, Colombia, and PUR: Puerto Ricans from Puerto Rico) were close to European populations, which in general is consistent with human migration and admixture history.

In spite of the very limited sample sizes, HWE and LD were tested. It was found that 51 out of 520 macrohaplotypes had p-values <0.05 for HWE tests. After Bonferroni correction (p-value<0.000096; 0.05/520), only 5 macrohaplotypes were still significantly departing from HWE. 347 out of 4,940 macrohaplotype pairs had p-values <0.05 for LD tests. After Bonferroni correction (p-value<1×10⁻⁵; 0.05/520), only 2 macrohaplotype pairs were still in LD. These significances may be due to the very limited sample sizes of the populations (only 61˜113 samples for each of these 26 populations) and that the macrohaplotypes are extremely polymorphic.

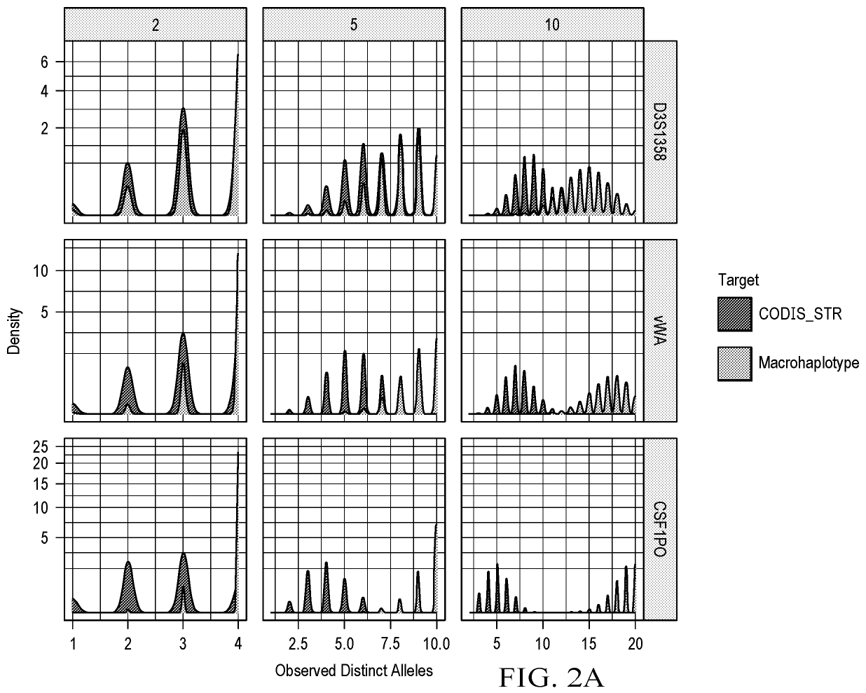

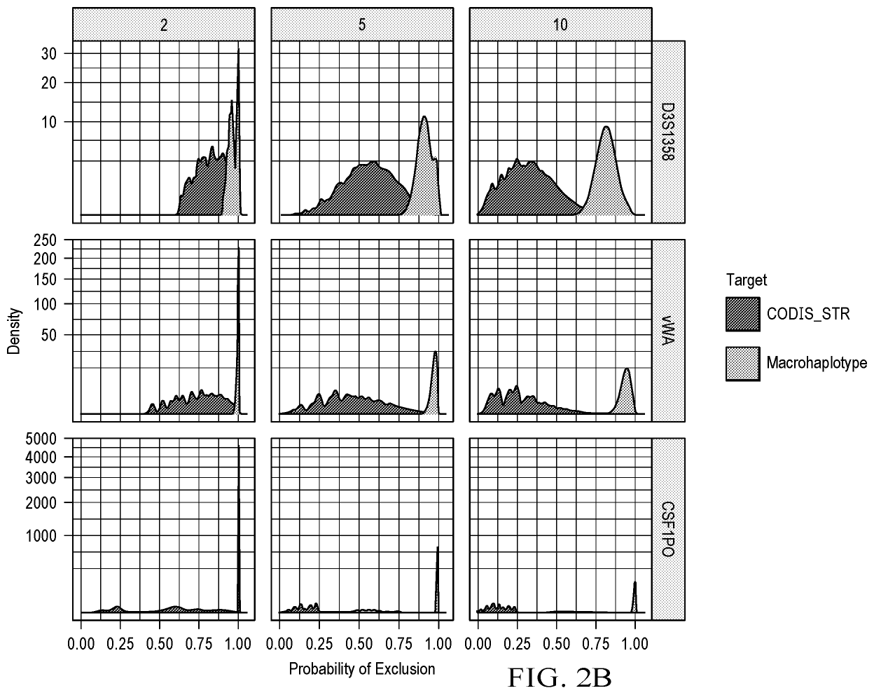

Because of the evident increased discrimination power, the capabilities of macrohaplotypes for mixture interpretation were evaluated. Compared with the CODIS STRs only, the chances to observe overlapped haplotypes of the macrohaplotypes were much lower. On average, 3.87 distinct haplotypes, with a standard error (SE) of 0.02, were observed for a two-person mixture with the macrohaplotypes; in contrast, 3.12 distinct alleles (SE=0.07) were observed on average for a two-person mixture with CODIS STRs only. The average homozygosity of the macrohaplotypes is 2.2%, thus, the chance to observe haplotypes overlapping between two individuals is very low. The differences were more noticeable for mixtures with a higher number of contributors (NOC). For a ten-person mixture, on average, 17.06 distinct haplotypes (SE=0.35) were observed with a macrohaplotype, while only 7.49 distinct observed alleles (SE=0.53) were observed with a CODIS STR. Further, FIG. 2A shows the distributions of the number of observed distinct alleles for 2, 5, and 10 persons mixtures at D3S1358, vWA, and CSF1PO, which represent the highest, geometric mean, and lowest RMP differences between the CODIS STRs and the associated macrohaplotypes. Even for the macrohaplotype with the least improved RMP (i.e., D3S1358), much higher numbers of distinct alleles were observed with the macrohaplotype compared with the CODIS STR, particularly for mixtures with higher NOC. The distribution differences were further widened for the macrohaplotypes with lowered RMPs (e.g., vWA and CSF1PO). Apparently, the macrohaplotypes can better estimate the NOC of the mixtures compared with the CODIS STRs, particularly for mixtures with high NOC.

In addition, the PEs of macrohaplotypes also were substantially higher than those of CODIS STRs, particularly for mixtures with a high NOC. For two-person mixtures, the average PEs were 98.9% (SE=0.3%) and 73.1% (SE=3.3%) for macrohaplotypes and CODIS STR only, respectively. For ten-person mixtures, the average PE of macrohaplotypes was 91.7% (SE=1.6%), while the average PE of CODIS STRs was only 25.7% (SE=3.6%). In other words, one macrohaplotype is equivalent to 3˜8 CODIS STRs in terms of the capability of excluding non-contributors, depending on the NOC in the mixtures. Further, FIG. 2B shows the distributions of PEs for 2, 5, and 10 persons mixtures at D3S1358, vWA, and CSF1PO. Even for the macrohaplotype with the least improved RMP (i.e., D3S1358), 99.8% of the 2-person mixtures had PE >0.9, while if only the CODIS STR was used, this percentage reduced to 23.5%. Same as the distributions of the observed distinct alleles, the distribution differences were further widened for the macrohaplotypes with lowered RMPs and mixtures with higher NOC. The macrohaplotype of CSF1PO alone could exclude >99.1% of the populations even with 10-person mixtures. Thus, macrohaplotypes substantially outperform CODIS STRs for interpreting mixtures, particularly for mixtures with a high number of contributors.

In this study, a novel forensic marker, macrohaplotype is described, which combines a CODIS STR and flanking variants in an extended fashion. With the capabilities of long-read sequencing technologies and the fact that some forensic mixtures may contain relatively intact DNA, 20 optimal macrohaplotypes with a size of 8 k bp were designed based on an updated SNP+STR panel to offer extremely high numbers of haplotypes and very high discrimination power on a per marker basis. On average, there were 30-times more haplotypes in the macrohaplotypes than the number of alleles in the CODIS STRs. The average RMP per macrohaplotype was two magnitudes higher than that of CODIS STRs. With macrohaplotypes, the chance of observing allele overlap among different contributors would be substantially reduced over current CODIS STRs' capabilities, which would provide higher accuracy in determining the number of contributors in a sample, increase the chance to exclude a non-contributor, and improve mixture deconvolution. Indeed, the macrohaplotypes are compatible with a likelihood ratio (LR) based mixture interpretation methods, but with a higher power of observing the DNA evidence for the support of different hypotheses. The macrohaplotypes are much more informative compared with other compound markers, but more importantly are backwards compatible with the CODIS core STR loci used in many national DNA databases.

This study used a size of 8 k bp for illustrative purposes. The actual sizes of macrohaplotypes may be decided (and designed) dependent on the sizes of the extracted DNA fragments, which can be determined by measuring DNA fragment sizes (e.g., Agilent TapeStation) or developing an assay similar to current quantitative PCR assays but with a range of larger amplicons. Following the size measurement, a triage could be performed, and an assay could be selected that is compatible with the quality of the DNA evidence. Therefore, different sizes of DNA fragments (e.g., ˜8 k, ˜4 k, ˜2 k, ˜1 k bp) could be considered, together with their impact on discrimination power based on reducing the sizes of the fragments. Regardless, a partial profile of only a few macrohaplotypes could be quite informative, especially from an exclusionary perspective. Thus, macrohaplotypes can be used with the slightly or moderately degraded samples, since meaningful interpretation may be obtained by just a few detected macrohaplotypes. High LRs also could be obtained supporting a contributor hypothesis, as the discrimination power of a single macrohaplotype may be equivalent to that of 3 sequence-based STRs, while providing a very low adventitious LR rate for non-contributors.

These CODIS STRs in the Saini's panel were called by HipSTR and Tredparse [37, 43, 44], which are general STR callers and may not follow the forensic standards to call the STR alleles. Therefore, the accuracies of the CODIS STR sequences in the current macrohaplotypes may be improved with forensically generated sequences, either from existing data (e.g., Aalbers et al. [45]) or re-sequencing these samples by the forensically designed kits. In addition, the phase information of the 1000 genomes data and the Saini's SNP+STR panel was statistically imputed as SRS technologies were used to generate the data. However, the present invention can be used with LRS technologies that can readily sequence long DNA fragments (e.g., >15 k bp) to provide complete phasing of the target regions. Together with the substantially improved sequencing accuracies of the LRS technologies, which have reached the same level of accuracies of the SRS technologies [46], the haplotypes in the macrohaplotypes using the present invention can be more precisely determined.

With a large number of variants included, single or a very few macrohaplotypes perform similarly with that of the lineage markers, such as Y chromosome STR haplotypes [47], which are considered extremely polymorphic single haplotype systems in their own right. Some macrohaplotypes (e.g., TH01 and TPOX) included more variants than the other macrohaplotypes, but still had lower discrimination power. This observation is likely due to the discrimination powers of many SNVs in these macrohaplotypes were relatively low, and many SNVs were in LD. Thus, some SNVs may be pruned in terms of LD to reduce the number of SNVs in the macrohaplotypes (and possibly the length), but with no or little impact on discrimination power. The sample sizes in this study were small. More haplotypes would be observed by typing more samples, and the haplotype frequencies can be more precisely estimated with more samples. A recent study also showed that the number of variants was substantially underestimated [48]. Namely, more than 410 million SNVs were observed in 53,831 individuals, and 78.7% of which had not been reported previously. In addition, using LRS technologies with the present invention will enable detection of more variants compared with the SRS technologies, since whole macrohaplotype(s) can be sequenced without any gap in the target region. Thus, more variants, particularly the private variants, may be included in macrohaplotypes with LRS technologies and applicable variant calling algorithms.

Although these macrohaplotypes were designed to enhance mixture interpretation, these markers also can be used for various forensic applications, such as single source sample identification, kinship analysis, cell line verification, etc. Particularly, for kinship analysis, in addition to the high discrimination powers, the macrohaplotypes can address potential STR mutations in kinship cases by evaluating variants in the flanking region, which in turn could reduce the effect of STR mutations on the LR calculation.

Thus, the present invention includes the development and use of macrohaplotypes for improving mixture interpretation in applicable casework, but also demonstrates the power of these markers for other forensic applications. Given the results reported herein and studies that have been conducted on the LRS sequencing technologies for calling STR alleles [49, 50], efforts to build a complete workflow, both wet-lab and bioinformatics, using the present invention, to provide a robust method that accurately calls the variants and generate the haplotypes of the macrohaplotypes.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Gill P, Jeffreys A J, Werrett D J (1985) Forensic application of DNA ‘fingerprints’. Nature 318: 577-9.
2. Voorhees J C, Ferrance J P, Landers J P (2006) Enhanced elution of sperm from cotton swabs via enzymatic digestion for rape kit analysis. J Forensic Sci 51: 574-9. doi: 10.1111/j.1556-4029.2006.00112.x
3. Giusti A, Baird M, Pasquale S, Balazs I, Glassberg J (1986) Application of deoxyribonucleic acid (DNA) polymorphisms to the analysis of DNA recovered from sperm. Journal of Forensic Science 31: 409-17.
4. Vandewoestyne M, Van Nieuwerburgh F, Van Hoofstat D, Deforce D (2012) Evaluation of three DNA extraction protocols for forensic STR typing after laser capture microdissection. Forensic Sci Int Genet 6: 258-62. doi: 10.1016/j.fsigen.2011.06.002
5. Šafařik I, Šafařiková M (1999) Use of magnetic techniques for the isolation of cells. Journal of Chromatography B: Biomedical Sciences and Applications 722: 33-53.
6. Buoncristiani M R, Timken M D (2009) Development of a procedure for dielectrophoretic (DEP) separation of sperm and epithelial cells for application to sexual assault case evidence. Bureau of Justice Statistics.
7. Gill P, Brenner C H, Buckleton J S et al (2006) DNA commission of the International Society of Forensic Genetics: Recommendations on the interpretation of mixtures. Forensic Sci Int 160: 90-101. doi: 10.1016/j.forsciint.2006.04.009.
8. SWGDAM (2015) Guidelines for the Validation of Probabilistic Genotyping Systems.
9. Bright J A, Taylor D, McGovern C et al (2016) Developmental validation of STRmix, expert software for the interpretation of forensic DNA profiles. Forensic Sci Int Genet 23: 226-39. doi: 10.1016/j.fsigen.2016.05.007
10. Gill P, Haned H, Eduardoff M, Santos C, Phillips C, Parson W (2015) The open-source software LRmix can be used to analyse SNP mixtures. Forensic Sci Int Genet Suppl Ser 5: e50-e1.
11. Perlin M W, Legler M M, Spencer C E et al (2011) Validating TrueAllele® DNA mixture interpretation. J Forensic Sci 56: 1430-47.
12. Bleka O, Storvik G, Gill P (2016) EuroForMix: An open source software based on a continuous model to evaluate STR DNA profiles from a mixture of contributors with artefacts. Forensic Sci Int Genet 21: 35-44. doi: https://doi.org/10.1016/j.fsigen.2015.11.008.
13. Ge J, Budowle B, Planz J V, Chakraborty R (2010) Haplotype block: a new type of forensic DNA markers. Int J Legal Med 124: 353-61. doi: 10.1007/s00414-009-0400-5
14. Kidd K K, Speed W C, Pakstis A J et al (2017) Evaluating 130 microhaplotypes across a global set of 83 populations. Forensic Sci Int Genet 29: 29-37. doi: 10.1016/j.fsigen.2017.03.014
15. Castella V, Gervaix J, Hall D (2013) DIP-STR: Highly Sensitive Markers for the Analysis of Unbalanced Genomic Mixtures. Human Mutation 34: 644-54. doi: 10.1002/humu.22280
16. Wang L, He W, Mao J et al (2015) Development of a SNP-STRs multiplex for forensic identification. Forensic Sci Int Genet Suppl Ser 5: e598-e600. doi: 10.1016/j.fsigss.2015.09.236
17. Liu Z, Liu J, Wang J et al (2018) A set of 14 DIP-SNP markers to detect unbalanced DNA mixtures. Biochem Biophys Res Commun 497: 591-6. doi: 10.1016/j.bbrc.2018.02.109
18. Voskoboinik L, Darvasi A (2011) Forensic identification of an individual in complex DNA mixtures. Forensic Sci Int Genet 5: 428-35. doi: 10.1016/j.fsigen.2010.09.002
19. Voskoboinik L, Ayers S B, LeFebvre A K, Darvasi A (2015) SNP-microarrays can accurately identify the presence of an individual in complex forensic DNA mixtures. Forensic Sci Int Genet 16: 208-15. doi: 10.1016/j.fsigen.2015.01.009
20. Gill P, Phillips C, McGovern C, Bright J A, Buckleton J (2012) An evaluation of potential allelic association between the STRs vWA and D12S391: implications in criminal casework and applications to short pedigrees. Forensic Sci Int Genet 6: 477-86. doi: 10.1016/j.fsigen.2011.11.001
21. Epstein M P, Duren W L, Boehnke M (2000) Improved inference of relationship for pairs of individuals. Am J Hum Genet 67: 1219-31. doi: 10.1016/S0002-9297(07)62952-8
22. Homer N, Szelinger S, Redman M et al (2008) Resolving individuals contributing trace amounts of DNA to highly complex mixtures using high-density SNP genotyping microarrays. PLoS Genet 4.
23. Egeland T, Fonnelop A E, Berg P R, Kent M, Lien S (2012) Complex mixtures: a critical examination of a paper by Homer et al. Forensic Sci Int Genet 6: 64-9. doi: 10.1016/j.fsigen.2011.02.003
24. Borsting C, Morling N (2015) Next generation sequencing and its applications in forensic genetics. Forensic Sci Int Genet 18: 78-89.
25. Novroski N M M, King J L, Churchill J D, Seah L H, Budowle B (2016) Characterization of genetic sequence variation of 58 STR loci in four major population groups. Forensic Sci Int Genet 25: 214-26. doi: 10.1016/j.fsigen.2016.09.007.
26. Van Neste C, Van Nieuwerburgh F, Van Hoofstat D, Deforce D (2012) Forensic STR analysis using massive parallel sequencing. Forensic Sci Int Genet.
27. Bornman D M, Hester M E, Schuetter J M et al (2012) Short-read, high-throughput sequencing technology for STR genotyping. Biotech Rapid Dispatches 2012: 1-6.
28. Jeffreys A J, Wilson V, Them S L (1985) Hypervariable ‘minisatellite’ regions in human DNA. Nature 314: 67-73.
29. Cornelis S, Willems S, Van Neste C et al (2018) Forensic STR profiling using Oxford Nanopore Technologies' MinION sequencer. doi: 10.1101/433151.
30. Lindberg M R, Schmedes S E, Hewitt F C et al (2016) A Comparison and Integration of MiSeq and MinION Platforms for Sequencing Single Source and Mixed Mitochondrial Genomes. PLoS One 11: e0167600. doi: 10.1371/journal.pone.0167600.
31. Zaaijer S, Gordon A, Speyer D, Piccone R, Groen S C, Erlich Y (2017) Rapid re-identification of human samples using portable DNA sequencing. Elife 6. doi: 10.7554/eLife.27798.
32. Mitsuhashi S, Kryukov K, Nakagawa S et al (2017) A portable system for rapid bacterial composition analysis using a nanopore-based sequencer and laptop computer. Scientific reports 7: 1-9.
33. Plesivkova D, Richards R, Harbison S (2019) A review of the potential of the MinION™ single-molecule sequencing system for forensic applications. Wiley Interdisciplinary Reviews: Forensic Science 1. doi: 10.1002/wfs2.1323.
34. Scheet P, Stephens M (2006) A fast and flexible statistical model for large-scale population genotype data: applications to inferring missing genotypes and haplotypic phase. Am J Hum Genet 78: 629-44. doi: 10.1086/502802.
35. Browning B L, Zhou Y, Browning S R (2018) A One-Penny Imputed Genome from Next-Generation Reference Panels. Am J Hum Genet 103: 338-48. doi: 10.1016/j.ajhg.2018.07.015
36. Midha M K, Wu M, Chiu K P (2019) Long-read sequencing in deciphering human genetics to a greater depth. Hum Genet 138: 1201-15. doi: 10.1007/s00439-019-02064-y.
37. Saini S, Mitra I, Mousavi N, Fotsing S F, Gymrek M (2018) A reference haplotype panel for genome-wide imputation of short tandem repeats. Nat Commun 9: 4397. doi: 10.1038/s41467-018-06694-0.
38. Consortium G P (2015) A global reference for human genetic variation. Nature 526: 68-74.
39. Phillips C, Gettings K B, King J L et al (2018) “The devil's in the detail”: Release of an expanded, enhanced and dynamically revised forensic STR Sequence Guide. Forensic Sci Int Genet 34: 162-9. doi: 10.1016/j.fsigen.2018.02.017.
40. Kidd K K, Speed W C (2015) Criteria for selecting microhaplotypes: mixture detection and deconvolution. Investig Genet 6: 1. doi: 10.1186/s13323-014-0018-3
41. Excoffier L, Lischer H E (2010) Arlequin suite ver 3.5: a new series of programs to perform population genetics analyses under Linux and Windows. Molecular ecology resources 10: 564-7.
42. Team R C (2017) R Core Team (2017). R: A language and environment for statistical computing. R Found Stat Comput Vienna, Austria.
43. Tang H, Kirkness E F, Lippert C et al (2017) Profiling of Short-Tandem-Repeat Disease Alleles in 12,632 Human Whole Genomes. Am J Hum Genet 101: 700-15. doi: 10.1016/j.ajhg.2017.09.013
44. Willems T, Zielinski D, Yuan J, Gordon A, Gymrek M, Erlich Y (2017) Genome-wide profiling of heritable and de novo STR variations. Nat Methods 14: 590-2. doi: 10.1038/nmeth.4267.
45. Aalbers S E, Weir B S (2020) Analyzing population structure for forensic STR markers in next generations sequencing data. Forensic Sci Int Genet. doi: 10.1016/j.fsigen.2020.102364.
46. Karst S M, Ziels R M, Kirkegaard R H et al (2021) High-accuracy long-read amplicon sequences using unique molecular identifiers with nanopore or PacBio sequencing. Nature Methods: 1-5.
47. Budowle B, van Daal A (2008) Forensically relevant SNP classes. Biotechniques 44: 603-8, 10. doi: 10.2144/000112806.
48. Taliun D, Harris D N, Kessler M D et al (2019) Sequencing of 53,831 diverse genomes from the NHLBI TOPMed Program. BioRxiv: 563866.
49. Tytgat O, Gansemans Y, Weymaere J, Rubben K, Deforce D, Van Nieuwerburgh F (2020) Nanopore Sequencing of a Forensic STR Multiplex Reveals Loci Suitable for Single-Contributor STR Profiling. Genes (Basel) 11. doi: 10.3390/genes11040381.
50. Asogawa M, Ohno A, Nakagawa S et al (2020) Human short tandem repeat identification using a nanopore-based DNA sequencer: a pilot study. J Hum Genet 65: 21-4. doi: 10.1038/s10038-019-0688-z.

1. A method for determining nucleic acid contributors to a biological sample or specimen from nucleic acids obtained from single cells in the biological sample or specimen by determining one or more macrohaplotypes, comprising the steps of:
obtaining or having obtained a biological sample or specimen;
generating amplicons or obtaining a sequence of amplicons from the biological sample or specimen to obtain two or more markers selected from Short Tandem Repeat (STR), Single Nucleotide Polymorphisms (SNPs), Insertion-Deletions (Indels), or combinations thereof, from a paternal, maternal, or both chromosomes;
calculating from the one or more macrohaplotypes one or more nucleic acid contributors to the biological sample or specimen;
comparing the one or more macrohaplotypes to a reference or known macrohaplotype profile from a subject suspected of contributing nucleic acids to the biological sample or specimen; and
identifying a number of contributors to the biological sample or specimen.

2. The method of claim 1, wherein the step of generating amplicons is by long-read sequencing.

3. The method of claim 1, wherein the amplicons are 100, 500, 1000, 2000, 3000, 4000, 5000, 6000, 7000, 8000, 10000, 50000, 100000 or more base pairs.

4. The method of claim 1, further comprising at least one of: determining one or more macrohaplotypes from the markers on a paternal or a maternal chromosome;
comparing the one or more macrohaplotypes to a database of one or more nucleic acid-based forensic criminal databases and generating a list of investigative leads, an indictment document, or both; or
determining using a probabilistic mixture model using one or more processors one or more genotypes of the one or more contributors at the one or more macrohaplotypes.

5. (canceled)

6. The method of claim 1, wherein the macrohaplotype is further defined as a haplotype of a plurality of alleles determined from a plurality of markers on a paternal or a maternal chromosome; a haplotype of a plurality of alleles determined from all the markers on a paternal or a maternal chromosome, or both.

7.-8. (canceled)

9. The method of claim 1, wherein at least one of: the one or more nucleic acid contributors comprise 2, 3, 4, 5, 6, 7, 8, 9, 10 or more or more contributors;
the biological sample or specimen comprises DNA molecules or RNA molecules;
the biological sample or specimen comprises nucleic acid from zero, one, or more contaminant genomes and one genome of interest; or
the biological sample or specimen comprises cellular DNA.

10.-12. (canceled)

13. The method of claim 1, wherein the one or more macrohaplotypes comprise at least one of the Short Tandem Repeat (STR), Single Nucleotide Polymorphisms (SNPs), and Insertion-Deletions (Indels).

14. The method of claim 1, wherein the macrohaplotype is sequenced using a forward and a reverse primer selected from SEQ ID NOS: 1 to 40.

15. A method, implemented at a computer system that includes one or more processors and system memory, of quantifying a nucleic acid sample comprising nucleic acid of one or more contributors from one or more macrohaplotypes, the method comprising:
obtaining or having obtained a biological sample or specimen;
generating amplicons or obtaining a sequence of amplicons from the biological sample or specimen to obtain two or more markers selected from Short Tandem Repeat (STR), Single Nucleotide Polymorphisms (SNPs), Insertion-Deletions (Indels), or combinations thereof, from a paternal, maternal, or both chromosomes;
calculating, with the one or more processors, from the one or more macrohaplotypes one or more nucleic acid contributors to the biological sample or specimen;
comparing, with the one or more processors, the one or more macrohaplotypes to a reference or known macrohaplotype profile from a subject suspected of contributing nucleic acids to the biological sample or specimen; and
identifying, with the one or more processors, one or more contributors to the biological sample by quantifying, using a probabilistic mixture model and the one or more processors, one or more fractions of nucleic acid of the one or more contributors in the nucleic acid sample, wherein using the probabilistic mixture model comprises deconvolution of nucleic acid mixtures from a complex mixture of two or more nucleic acid contributors.

16. The method of claim 15, further comprising determining using a probabilistic mixture model and the one or more processors, one or more genotypes of the one or more contributors at the one or more macrohaplotypes.

17. The method of claim 15, wherein at least one of: the one or more nucleic acid contributors comprise 2, 3, 4, 5, 6, 7, 8, 9, 10 or more or more contributors;
the biological sample or specimen comprises DNA molecules or RNA molecules;
the biological sample or specimen comprises nucleic acid from zero, one, or more contaminant genomes and one genome of interest; or
the biological sample or specimen comprises cellular DNA.

18.-20. (canceled)

21. The method of claim 15, wherein the one or more macrohaplotypes comprise at least one of the Short Tandem Repeat (STR), Single Nucleotide Polymorphisms (SNPs), and Insertion-Deletions (Indels).

22. The method of claim 15, wherein the step of generating amplicons is by long-read sequencing.

23. The method of claim 15, wherein the amplicons are 100, 500, 1000, 2000, 3000, 4000, 5000, 6000, 7000, 8000, 10000, 50000, 100000 or more base pairs.

24. The method of claim 15, further comprising at least one of determining one or more macrohaplotypes from the markers on the same paternal or maternal chromosome;
comparing the one or more macrohaplotypes to a database of one or more nucleic acid-based forensic criminal databases and generating a list of investigative leads, an indictment document, or both; or
determining using a probabilistic mixture model using one or more processors one or more genotypes of the one or more contributors at the one or more macrohaplotypes.

25. (canceled)

26. The method of claim 15, wherein the macrohaplotype is further defined as a haplotype of a plurality of alleles determined from a plurality of markers on the same paternal or maternal chromosome; a haplotype of a plurality of alleles determined from all the markers on a paternal or a maternal chromosome, or both.

27. (canceled)

28. The method of claim 15, wherein the macrohaplotype is sequenced using a forward and a reverse primer selected from SEQ ID NOS: 1 to 40.

29. The method of claim 1, further comprising
comparing the one or more macrohaplotypes to a reference or known macrohaplotype profile from a subject suspected of contributing nucleic acids to the biological sample or specimen; and
identifying a number of contributors to the biological sample or specimen.

30. A method for method for generating sequences for one or more macrohaplotypes comprising the steps of:
(a) selecting one or more Short Tandem Repeat (STRs) (S) and a sequence length (L) of a predefined size;
(b) determining one or more polymorphisms in the sequence surrounding S with a Single Nucleotide Polymorphisms (SNPs) and STR panel with n polymorphisms on a left side and m polymorphisms on a right size of S;
(c) generating a list of possible macrohaplotypes with a size of L that contains S into a candidate list (Lm);
(d) using a sliding window algorithm for all possible macrohaplotype configurations, wherein a window slides one polymorphism at a time from left to right, wherein a polymorphism sliding change creates a new macrohaplotype with one or more different polymorphism(s);
(e) selecting the macrohaplotype with the lowest RMP on the candidate list (Lm); and
(f) repeating steps (a)-(e) for each STRs to generate a panel of optimal macrohaplotypes.

31. A kit for determining for determining nucleic acid contributors to a biological sample or specimen from nucleic acids by determining one or more macrohaplotypes, comprising:
a container comprising one or more primer pairs for detecting macrohaplotypes from two or more markers selected from Short Tandem Repeat (STR), Single Nucleotide Polymorphisms (SNPs), Insertion-Deletions (Indels), or combinations thereof and reagents generating amplicons or obtaining a sequence of amplicons from the biological sample or specimen to obtain two or more markers selected from Short Tandem Repeat (STR), Single Nucleotide Polymorphisms (SNPs), Insertion-Deletions (Indels), or combinations thereof, from a paternal, maternal, or both chromosomes and for sequencing amplified products with long range sequencing (LRS) to obtain sequence data;
instruction to:
call haplotype variants from the sequence data;
calculate from the one or more macrohaplotypes one or more nucleic acid contributors to the biological sample or specimen;
compare the one or more macrohaplotypes to a reference or known macrohaplotype profile from a subject suspected of contributing nucleic acids to the biological sample or specimen; and
identify a number of contributors to the biological sample or specimen.

32. The kit of claim 31, wherein the amplicons are 100, 500, 1000, 2000, 3000, 4000, 5000, 6000, 7000, 8000, 10000, 50000, 100000 or more base pairs.

33. The kit of claim 31, further comprising instructions for comparing the one or more macrohaplotypes to a database of one or more nucleic acid-based forensic criminal databases and generating a list of investigative leads, an indictment document, or both, instructions for determining using a probabilistic mixture model on one or more processors, one or more genotypes of the one or more contributors at the one or more macrohaplotypes, or both.

34. (canceled)

35. The kit of claim 31, wherein the reagents amplify DNA molecules or RNA molecules.

36. The kit of claim 31, wherein the macrohaplotype is sequenced using a forward and a reverse primer selected from SEQ ID NOS: 1 to 40.
